(19) United States
(12) Patent Application Publication
(10) Pub. No.: US 2018/0309907 A1
(43) Pub. Date: Oct. 25, 2018

(54) IMAGE PROCESSING APPARATUS AND IMAGING APPARATUS

(71) Applicant: Shimadzu Corporation, Kyoto (JP)

(72) Inventor: Hiroyuki Tsumatori, Kyoto (JP)

(73) Assignee: Shimadzu Corporation, Kyoto (JP)

(21) Appl. No.: 15/955,032

(22) Filed: Apr. 17, 2018

(30) Foreign Application Priority Data
Apr. 25, 2017 (JP) 2017-085808

(51) Int. Cl. H04N 5/217; H04N 5/225; H04N 5/232; H04N 5/77; A61B 5/00

(52) U.S. Cl. CPC H04N 5/217; H04N 5/2256; H04N 5/23293; H04N 5/77; A61B 2576/00; A61B 5/7203; A61B 5/725; A61B 5/742; A61B 5/0071

(57) ABSTRACT

A controller has an image processing unit that performs various types of image processing. The image processing unit has a memory unit that sets IDs by expressing a plurality of pixel values in a series of bits and stores computation results obtained by combining all of the pixel values by associating with the IDs in advance, and a reading unit that reads the computation result stored in the memory unit using the ID obtained by expressing the plurality of input pixel values in a series of bits.

### FIELD

The present invention relates to an image processing apparatus that performs computation using a plurality of pixel values or a plurality of indices associated with the pixel values, and an imaging apparatus including an excitation light source that irradiates excitation light for exciting a fluorescent dye injected into an examinee onto examinee, a camera that captures a fluorescent image by photographing fluorescence generated from the fluorescent dye by irradiating the excitation light, and an image processing unit that performs image processing for the fluorescent image captured by the camera and displays the image on a display unit.

### BACKGROUND

A technique called “near-infrared fluorescence imaging” has been employed in contrastradiography for a blood vessel or a lymphatic vessel in surgery. In this near-infrared fluorescence imaging, indocyanine green (ICG) as a fluorescent dye is administrated to a lesion by injecting it into an examinee's body using an injector or the like. In addition, as near-infrared light having a wavelength of 600 to 850 nm is irradiated onto this indocyanine green as excitation light, the indocyanine green generates near-infrared fluorescence having a wavelength of about 750 to 900 nm. This fluorescence is captured using an image sensor capable of detecting the near-infrared light, and an image thereof is displayed on a display unit such as a liquid crystal display panel. In this near-infrared fluorescence imaging, it is possible to observe a blood vessel or a lymphatic vessel present at a depth of about 20 mm from a body surface.

In recent years, a technique of fluorescently marking a tumor and using this mark in surgery navigation has been focused. As a fluorescent marking agent for fluorescently marking a tumor, 5-aminolevulinic acid (5-ALA) is employed. In a case where this 5-aminolevulinic acid (5-ALA) (hereinafter, referred to as “5-ALA”) is administrated to an examinee, the 5-ALA is metabolized by protoporphyrin IX (PpIX) which is the fluorescent dye. Note that this PpIX is specifically accumulated in a cancer cell. In addition, as visible light having a wavelength of about 410 nm is irradiated onto PpIX which is a metabolite of 5-ALA, red visible light having a wavelength of about 630 nm is emitted from PpIX as fluorescence. It is possible to check a cancer cell by capturing the fluorescence from the PpIX using an image sensor and observing it.

In an imaging apparatus that captures fluorescence from a fluorescent dye injected into a body, the camera captures weak fluorescence from the fluorescent dye. Therefore, it is necessary to increase sensitivity of the camera. In a case where the sensitivity of the camera increases in this manner, noise in the fluorescent image is also amplified. This degrades quality of the fluorescent image.

Patent Literatures 1 and 2 discuss noise rejection techniques using a recursive filter as a time filter. That is, Patent Literature 1 discusses an image processing method, an image processing apparatus, and a fluoroscopic apparatus, in which a recursive filter that performs weighted addition for the (N)th frame and the (N−1)th frame immediately previous to the (N)th frame of the fluorescent image. In addition, Patent Literature 2 discusses an image processing apparatus that obtains noise components and motion components of an image for each pixel by comparing image data of the current frame and image data of the previous frame and controls recursive filter coefficients for each pixel depending on the noise components and the motion components.

Patent Literature 3 discusses a noise rejection technique in which a bilateral filter is employed as a spatial filter.
[Patent Literature 1] JP-A-8-255238
[Patent Literature 2] JP-A-6-47035
[Patent Literature 3] JP-A-2014-27630

### SUMMARY

Regardless of whether the time filter such as the recursive filter or the spatial filter such as the bilateral filter is employed, it is necessary to execute computation for each pixel in order to perform noise rejection. Therefore, a predetermined period of time is necessary to perform the computation.

For example, in a case where the noise rejection is performed using the aforementioned recursive filter, a pixel value F_{N} of the output frame is computed on the basis of the following formula, assuming that a pixel value of the image captured by the camera is set as an input pixel value I_{N}, a pixel value of the (N−1)th frame is set as a reference pixel value F_{N-1}, and “k” denotes a weighting factor.

F_{N}=kI_{N}+(1−k)F_{N-1} [Formula 1]

In this case, it is necessary to perform a total of four computations for obtaining a product between the input pixel value and the weighting factor, a difference between “1” and the weighting factor, a product between the difference and the reference pixel value, a sum of the products, for overall pixels of an image resolution by applying the input pixel value I_{N} and the reference pixel value F_{N-1} to the aforementioned formula for each pixel.

In a case where the noise rejection is performed using the aforementioned bilateral filter, the output pixel value g is computed on the basis of the following formula, assuming that “f” denotes the input pixel value, “(i, j)” denotes X-Y coordinates, “w”, “m”, and “n” denote movement amounts, “σ” denotes a standard deviation, and a product of the exponential functions is set as a weighting factor.

$$g ( i , j ) = \frac{\begin{matrix}{\sum }_{n = - w}^{w} {\sum }_{m = - w}^{w} f ( i + m , j + n ) exp ( - \frac{{m}^{2} + {n}^{2}}{2 {σ}_{1}^{2}} ) \\ exp ( - \frac{\begin{matrix}( f ( i , j ) - \\ {f ( i + m , j + n ) )}^{2}\end{matrix}}{2 {σ}_{2}^{2}} )\end{matrix}}{{\sum }_{n = - w}^{w} {\sum }_{m = - w}^{w} exp ( - \frac{{m}^{2} + {n}^{2}}{2 {σ}_{1}^{2}} ) exp ( - \frac{\begin{matrix}( f ( i , j ) - \\ {f ( i + m , j + n ) )}^{2}\end{matrix}}{2 {σ}_{2}^{2}} )} \qquad [ Formula 2 ]$$

In this case, it is necessary to perform a total of forty computations, including fourteen computations for calculating a weighting factor by applying the input pixel value f(i, j) and the reference pixel value f(i+m, j+n) to the aforementioned formula for each pixel, seventeen computations for calculating a numerator, eight computations for calculating a denominator, one computation for dividing the numerator by the denominator, for overall pixels of an image resolution.

These computations can be processed in real time for a resolution currently and typically employed. However, in a case where the number of pixels increases as in 4K or 8K employed in recent years, it is difficult to perform the computations in real time. In particular, in a case where time is necessary in the image processing such as medical image processing in which it is necessary to display an image of an examinee in real time, a problem may occur in operation.

Such a problem similarly occurs in various types of image processing apparatuses that perform the computation using of a plurality of pixel values, including input pixel values and reference pixel values, in addition to noise rejection.

In view of the aforementioned problems, an object of the invention is to provide an image processing apparatus and an imaging apparatus capable of fast processing even when the image processing is performed for a high-resolution image.

According to the invention, there is provided an image processing apparatus that performs computation using a plurality of pixel values or a plurality of indices associated with the pixel values, the image processing apparatus including: a memory unit that sets the plurality of pixel values or the plurality of indices associated with the pixel values as IDs expressed in a series of bits, and stores computation results obtained by combining all of the pixel values or all of the indices by associating with the IDs in advance; and a reading unit that reads the computation result stored in the memory unit using the ID obtained by expressing the plurality of pixel values or the plurality of indices associated with the pixel values in a series of bits.

According to the invention, the image processing is to reject noise from an image captured by a camera using a noise rejection filter that performs computation using an input pixel value and a reference pixel value, and the reading unit reads the computation result stored in the memory unit using the ID obtained by expressing the input pixel value and the reference pixel value input at the time of the noise rejection in a series of bits.

According to the invention, an ID obtained by expressing reference information in addition to the input pixel value and the reference pixel value in a series of bits is used.

According to the invention, there is provided an imaging apparatus including: an excitation light source that irradiates excitation light for exciting a fluorescent dye injected into an examinee onto the examinee; a camera that obtains a fluorescent image by capturing fluorescence generated from the fluorescent dye by irradiating the excitation light; an image processing unit that performs image processing to display the fluorescent image obtained by the camera on a display unit, the image processing unit performing noise rejection for an image captured by the camera using a noise rejection filter that performs computation using an input pixel value and a reference pixel value; a memory unit that sets an ID by expressing the input pixel value and the reference pixel value in a series of bits and stores computation results obtained by combining all of the input pixel values and all of the reference pixel values by associating with the IDs in advance; and a reading unit that reads the computation result stored in the memory unit using the ID obtained by expressing the input pixel value and the reference pixel value input at the time of the noise rejection in a series of bits.

According to the invention, since the computation result stored by associating with ID in advance is read on the basis of the ID, it is possible to perform fast processing even when a high-resolution image is processed.

According to the invention, it is possible to perform fast processing even in a noise rejection process for a high-resolution image.

According to the invention, it is possible to read reference information along with the computation result.

According to the invention, even when noise generated by increasing sensitivity of a camera to capture weak fluorescence from a fluorescent dye using the camera is rejected, it is possible to perform fast noise rejection processing and display a fluorescent image in real time.

### BRIEF DESCRIPTION

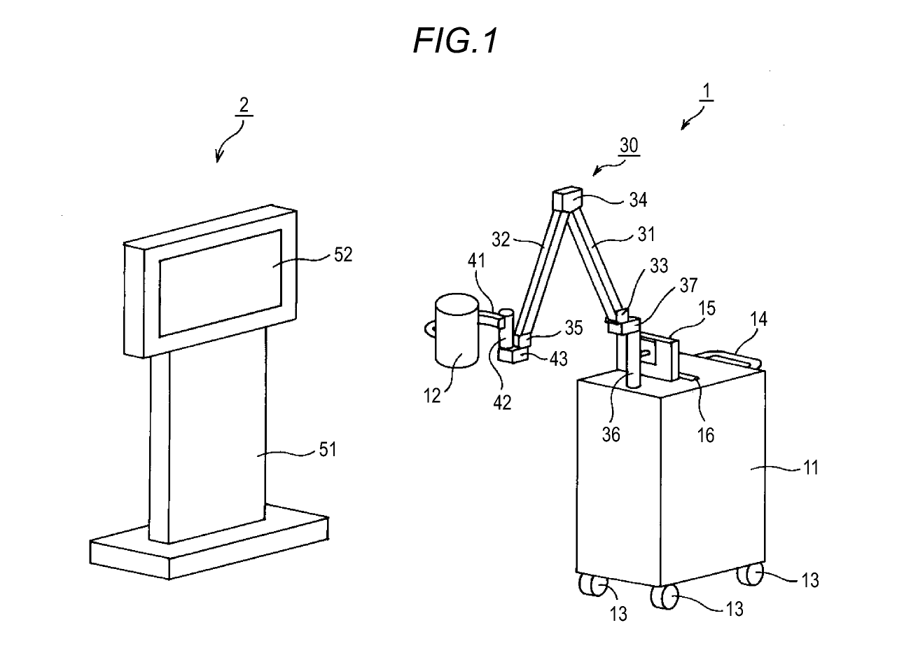

FIG. 1 is a perspective view illustrating an imaging apparatus 1 according to the invention along with a display device 2;

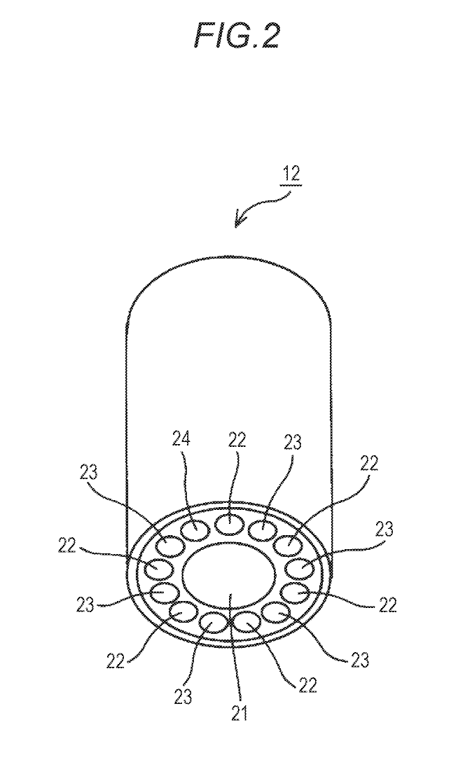

FIG. 2 is a schematic diagram illustrating an illumination and imaging unit 12;

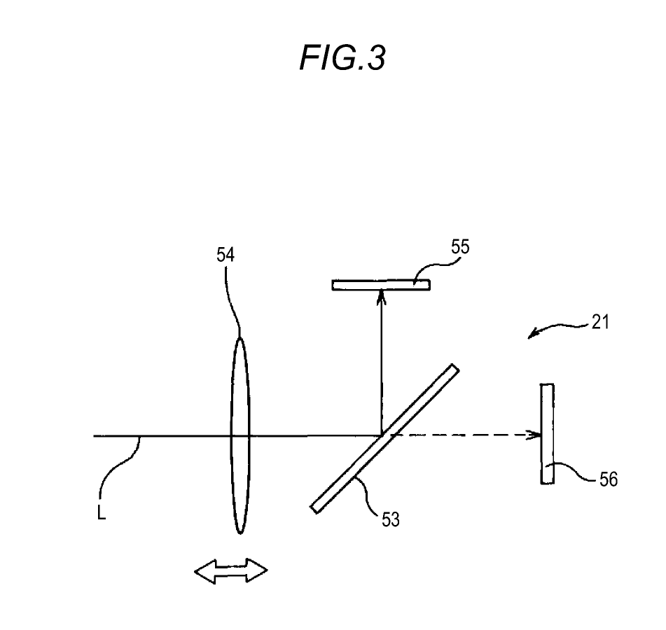

FIG. 3 is a schematic diagram illustrating a camera 21 of the illumination and imaging unit 12; and

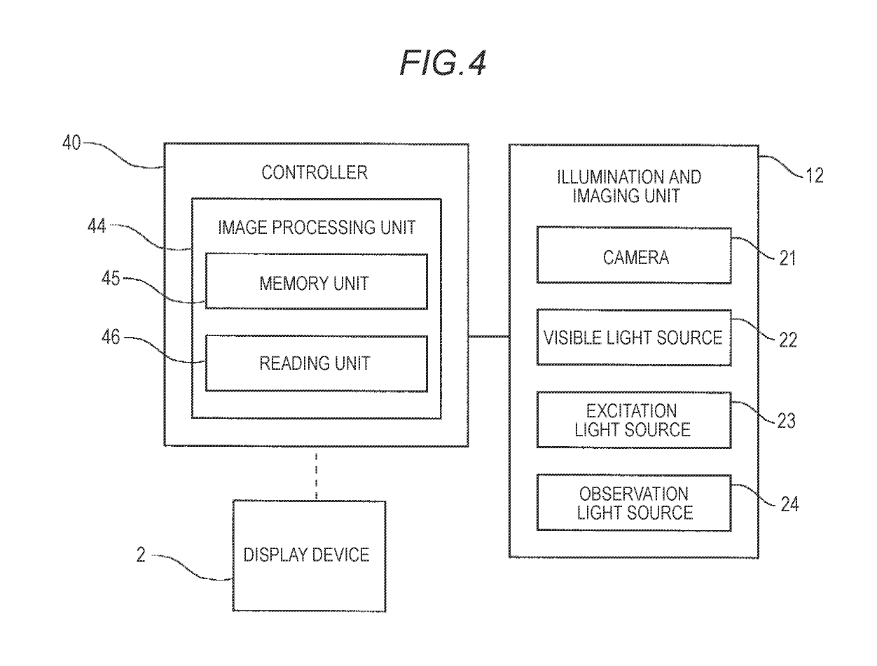

FIG. 4 is a block diagram illustrating a main control system of the imaging apparatus 1 according to the invention along with the display device 2.

### DETAILED DESCRIPTION

Embodiments of the invention will now be described with reference to the accompanying drawings. FIG. 1 is a perspective view illustrating an imaging apparatus 1 according to the invention along with a display device 2.

The display device 2 has a configuration in which a display unit 52 such as a large-sized liquid crystal display device is supported by a support mechanism 51.

The imaging apparatus 1 irradiates excitation light onto indocyanine green as a fluorescent dye injected into an examinee's body, captures fluorescence emitted from the indocyanine green, and displays the fluorescent image on the display device 2 along with a color image as a visible image of the examinee. In addition, the imaging apparatus 1 displays the fluorescent image and the color image described above and measures an intensity of the fluorescence in an region of interest of the examinee over time to obtain a time intensity curve (TIC) of the fluorescence in the region of interest of the examinee.

The imaging apparatus 1 includes a cart 11 having four wheels 13, an arm mechanism 30 arranged in the vicinity of a front side of a travel direction of the cart 11 on a top surface of the cart 11, an illumination and imaging unit 12 arranged in the arm mechanism 30 by interposing a subsidiary arm 41, and a monitor 15. A steering handle 14 used to drive the cart 11 is arranged in the rear side of the travel direction of the cart 11. In addition, a hollow 16 for mounting a manipulation unit (not shown) used to remotely manipulate the imaging apparatus 1 is provided on the top surface of the cart 11.

The arm mechanism 30 described above is arranged in the front side of the travel direction of the cart 11. The arm mechanism 30 has a first arm member 31 connected by a hinge 33 to a support portion 37 arranged in a post 36 erected in the front side of the travel direction of the cart 11. The first arm member 31 can be swayed with respect to the cart 11 by interposing the post 36 and the support portion 37 by virtue of the hinge 33. Note that the aforementioned monitor 15 is attached to the post 36.

A second arm member 32 is connected to the upper end of the first arm member 31 by a hinge 34. The second arm member 32 is swayable with respect to the first arm member 31 by virtue of the hinge 34. For this reason, the first and second arm members 31 and 32 illustrated in FIG. 1 can take a photographing posture in which the first and second arm members 31 and 32 are opened at a predetermined angle with respect to the hinge 34 serving as a connecting portion between the first and second arm members 31 and 32 and a standby posture in which the first and second arm members 31 and 32 are arranged close to each other.

A support portion 43 is connected to the lower end of the second arm member 32 by a hinge 35. The support portion 43 is swayable with respect to the second arm member 32 by virtue of the hinge 35. A rotation shaft 42 is supported by the support portion 43. In addition, the subsidiary arm 41 that supports the illumination and imaging unit 12 is pivoted against the rotation shaft 42 arranged in the distal end of the second arm member 32. For this reason, the illumination and imaging unit 12 moves, by virtue of pivoting of the subsidiary arm 41, between a front-side position of the travel direction of the cart 11 with respect to the arm mechanism 30 for taking a photographing posture or a standby posture illustrated in FIG. 1 and a rear-side position of the travel direction of the cart 11 with respect to the arm mechanism 30 which is a posture to move the cart 11.

FIG. 2 is a schematic diagram illustrating the illumination and imaging unit 12.

The illumination and imaging unit 12 has a camera 21 having a plurality image sensors capable of detecting a near-infrared ray and visible light as described below, a visible light source 22 having six LEDs arranged in an outer periphery of the camera 21, an excitation light source 23 having six LEDs, and an observation light source 24 having a single LED. The visible light source 22 irradiates visible light. The excitation light source 23 irradiates near-infrared light having a wavelength of 760 nm which is excitation light for exciting indocyanine green. In addition, the observation light source 24 irradiates near-infrared light having a wavelength of 810 nm approximate to the wavelength of the fluorescence generated from the indocyanine green. Note that the wavelength of the excitation light source 23 is not limited to 760 nm, and may be any wavelength as long as it can excite the indocyanine green. The wavelength of the observation light source 24 is not limited to 810 nm, and may be equal to or longer than the wavelength emitted from the indocyanine green.

FIG. 3 is a schematic diagram illustrating the camera 21 of the illumination and imaging unit 12.

The camera 21 has a movable lens 54 that reciprocates for focusing, a wavelength selection filter 53, a visible light image sensor 55, and a fluorescence image sensor 56. The visible light image sensor 55 and the fluorescence image sensor 56 are CMOS or CCD sensors. Note that, as the visible light image sensor 55, a sensor capable of capturing a visible light image as a color image is employed.

The visible light and the fluorescence incident to the camera 21 coaxially along an optical axis L pass through the movable lens 54 included in a focusing mechanism and then arrive at the wavelength selection filter 53. Out of the visible light and the fluorescence incident coaxially, the visible light is reflected on the wavelength selection filter 53 and is incident to the visible light image sensor 55. In addition, out of the visible light and the fluorescence incident coaxially, the fluorescence passes through the wavelength selection filter 53 and is incident to the fluorescence image sensor 56. In this case, by virtue of the focusing mechanism including the movable lens 54, the visible light is focused onto the visible light image sensor 55, and the fluorescence is focused onto the fluorescence image sensor 56. The visible light image sensor 55 captures a visible image as a color image at a predetermined frame rate. Furthermore, the fluorescence image sensor 56 captures a fluorescent image as a near-infrared image at a predetermined frame rate.

FIG. 4 is a block diagram illustrating a main control system of the imaging apparatus 1 according to the invention along with the display device 2.

The imaging apparatus 1 has a controller 40 having a CPU as a processor for executing logic operation, a ROM for storing operation programs necessary to control the apparatus, and a random access memory (RAM) for temporarily storing data or the like at the time of control to control the apparatus as a whole. The controller 40 is connected to the display device 2 described above. In addition, the controller 40 is connected to the illumination and imaging unit 12 having the camera 21, the visible light source 22, the excitation light source 23, and the observation light source 24.

The controller 40 has an image processing unit 44 that executes various types of image processing. The image processing unit 44 has a memory unit 45 that sets an ID by expressing an input pixel value and a reference pixel value in a series of bits and stores computation results obtained by combining all of the input pixel values and all of the reference pixel values by associating with the IDs in advance, and a reading unit 46 that reads the computation results stored in the memory unit 45 using the IDs obtained by expressing the input pixel values and the reference pixel values, which have been input, in a series of bits.

In a case where surgery for an examinee is performed using the imaging apparatus 1 having such a configuration, first, the observation light source 24 of the illumination and imaging unit 12 is turned on, and an image of the irradiation region is captured using the camera 21. The near-infrared light irradiated from the observation light source 24 at a wavelength of 810 nm approximate to the wavelength of the fluorescence generated from indocyanine green is not visually recognizable by human eyes. Meanwhile, in a case where near-infrared light having a wavelength of 810 nm is irradiated from the observation light source 24, and the image of the irradiation region is captured by the camera 21 by assuming that the camera 21 is normally operated, the image of the irradiation region of the near-infrared light is captured by the camera 21, and the image is displayed on the display unit 52 of the display device 2. As a result, it is possible to easily check the operation of the camera 21.

Then, indocyanine green is injected into an examinee using a syringe. In addition, near-infrared rays are irradiated from the excitation light source 23 of the illumination and imaging unit 12 toward a lesion of an examinee's organ, and visible light is irradiated from the visible light source 22. Note that, as the near-infrared light irradiated from the excitation light source 23, near-infrared light having a wavelength of 760 nm serving as excitation light to generate fluorescence from the indocyanine green as described above is employed. As a result, the indocyanine green injected into the examinee's body generates fluorescence within a near-infrared range having a peak at about 800 nm.

The camera 21 of the illumination and imaging unit 12 captures the vicinity of the lesion inside the examinee's organ at a predetermined frame rate. This camera 21 can detect near-infrared light and visible light as described above. The near-infrared image and the color image captured by the camera 21 at a predetermined frame rate are converted by the image processing unit 44 into image data that can be displayed on the display unit 52 of the display device 2 and are displayed on the display unit 52. In addition, the image processing unit 44 creates a synthetic image by synthesizing the color image and the near-infrared image using the near-infrared image data and the color image data according to need.

In a case where the fluorescence from the indocyanine green injected into an examinee's body is captured by the fluorescence image sensor 56 of the camera 21 of the imaging apparatus 1, it is necessary to capture weak fluorescence from the indocyanine green. Therefore, the fluorescence image sensor 56 is required to have high sensitivity. In a case where the sensitivity of the fluorescence image sensor 56 is set to be high in this manner, the noise of the fluorescent image in the time domain also increases, and this degrades quality of the fluorescent image. For this reason, the image processing unit 44 of the imaging apparatus 1 according to the invention employs a configuration for rejecting noise.

In this image processing unit 44, the memory unit 45 sets an ID by expressing an input pixel value and a reference pixel value in a series of bits, and stores computation results obtained by combining all of the input pixel values and all of the reference pixel values by associating with the IDs in advance. In addition, the reading unit 46 reads the computation results stored in the memory unit 45 using the IDs obtained by expressing the input pixel values and the reference pixel values, which have been input, in a series of bits.

This operation will now be described in more details. In the following description, for example, in a case where a recursive filter is employed as the time filter as described above, “F_{N}” is computed on the basis of a general formula of the recursive filter by assuming that “I_{N}” is set to “32”, and “Fi” is set to “30”.

In this case, assuming that the treated pixel values have a length of 8 bits, and two types of computations are performed, a computation result obtained by inputting “I_{N}” and “Fi” to the aforementioned formula can be expressed as a series of bits as follows, where “I” denotes a digit of a binary number corresponding to the input pixel value I_{N}, “F” denotes a digit of a binary number corresponding to the reference pixel value F_{N}, and “P” denotes a digit of a binary number corresponding to the processing type.

computation result=IIIIIIIIFFFFFFFFPP

As described above, assuming that “I_{N}” is set to “32” (corresponding to “00100000” as a binary notation of 8 bits), “Fi” is set to “30” (corresponding to “00011110” as a binary notation of 8 bits), a computation processing number is set to “1” (corresponding to “01” as a binary notation of 2 bits), and the computation result is “31”, the following formula can be obtained.

001000000001111001=31

Note that, in the aforementioned example, a case where “F_{N}” is computed on the basis of the aforementioned general formula of the recursive filter has been described by assuming that the recursive filter as a time filter is employed, “I_{N}” is set to “32”, and “Fi” is set to “30”. However, this may similarly apply to a case where a computation result g(i+m, j+m) is computed on the basis of the aforementioned general formula of the bilateral filter by assuming that the bilateral filter as a spatial filter is employed, the input pixel value f(i, j) is set to “32”, and the reference pixel value f(i+m, j+n) is set to “30”.

“I_{N}” and “Fi” are set as IDs expressed in a series of 18 bits, and a relationship between all values of “I_{N}” of 8 bits, all values of “Fi” of 8 bits, and all computation results obtained by applying “I_{N}” and “Fi” to the computation formula are stored in the memory unit 45 of the image processing unit 44.

The number of IDs becomes 18 bits (=262,144). For this reason, it is necessary to set a capacity of the memory unit 45 such that 262,144 pieces of information can be stored. However, in many cases, the computation results using “I_{N}” and “Fi” may become equal, or the computation result obtained by combining “I_{N}” and “Fi” may reach a maximum value or a minimum value. Therefore, in practice, the memory capacity does not necessarily increase to such a level.

When the image processing unit 44 executes the noise rejection process, the computation result stored in the memory unit 45 is read by the reading unit 46 of the image processing unit 44 by using the bits representing “I_{N}” and “Fi” input at the time of the noise rejection process and the bit representing the computation processing number as an ID. As a result, it is possible to obtain the same result as that obtained in a case where the computation is executed by applying “I_{N}” and “Fi” to the computation formula. For this reason, it is possible to obtain the same result as the computation result just by reading the computation result by using the bits representing the processing number of the computation of “I_{N}” and “Fi” as an ID even when the computation is complicated. For this reason, it is possible to execute the noise rejection process in real time.

It is possible to minimize computation cost regardless of complexity of the computation process and execute the noise rejection processing without generating a delay even in a high-resolution image. In this case, since the computation result is stored in advance, it is possible to prevent a rounding error that may be generated by hardware that executes the computation.

Note that the last 2 bits out of 18 bits of the ID described above are used to represent a processing type. This represents information, for example, regarding whether the recursive filter or the bilateral filter is employed. In addition, the last 2 bits may be used to represent information on a weighting factor “k” of the recursive filter or information on movement amounts “m” and “n” of the bilateral filter. In addition, in a case where the same processing is executed at all times, the last 2 bits may be omitted, and the remaining 16 bits of the ID may be used. Furthermore, if the number of the processing types exceeds “4”, a digit of 3 bits or more may be used.

Note that, in the aforementioned embodiment, the computation processing numbers of “I_{N}” and “Fi” are expressed as bits arranged in this order. However, this order may be arbitrarily set.

Although the computation processing numbers of “I_{N}” and “Fi” are expressed as bits arranged in this order in the aforementioned embodiment, they may be expressed as bits by arranging three or more pixel values. For example, (2n+1)² variables are necessary in the aforementioned bilateral filter. Therefore, in a case where the bilateral filter is employed, at least nine variables are expressed as bits by arranging them in sequence.

Although bits are expressed by arranging a plurality of pixel values in sequence in the aforementioned embodiment, a plurality of indices associated with the pixel values may be employed instead of a plurality of pixel values. That is, a mathematical function serving as an approximation of the computation result may be created, and variables of this approximation may be set as IDs, and the IDs may be stored by associating with the computation results of the approximation. For example, assuming functions “G(I, F)=x” and “H(I, F)=y” by setting the input pixel value “I” and the reference pixel value “F” as variables, the functions may be set as indices, and the indices associated with the pixel values may be used as IDs expressed in a series of bits.

In the aforementioned embodiment, the following formula is created and stored on the basis of a binary notation.

001000000001111001=31

Alternatively, the following formula may be created and stored on the basis of a decimal or hexadecimal notation. Note that, in the following formula, a radix of a base is expressed a lower right subscript.

32889₍₁₀₎=8079₍₁₆₎=31

In the aforementioned embodiment, the invention is applied to the imaging apparatus 1 that irradiates excitation light onto indocyanine green as a fluorescent dye injected to an examinee's body, captures fluorescence emitted from the indocyanine green, and displays the fluorescent image on the display device 2 along with a color image as a visible image of the examinee. Alternatively, the invention may also be applied to an image processing apparatus.

In the aforementioned embodiment, the invention is applied to an image processing apparatus that performs noise rejection for an image captured from the camera using a noise rejection filter that performs computation using the input pixel values and the reference pixel values. Alternatively, the invention may also be applied to various other types of image processing apparatuses that perform computation using the input pixel value and the reference pixel value. For example, the invention may also be applied to an image processing apparatus that synthesizes two images using the input pixel value and the reference pixel value. Furthermore, the invention may also be applied to an image processing apparatus that applies any effect to the input pixel value using the input pixel value and the reference pixel value.

1. An image processing apparatus that performs computation using a plurality of pixel values or a plurality of indices associated with the pixel values, the image processing apparatus comprising:
a memory unit that sets the plurality of pixel values or the plurality of indices associated with the pixel values as IDs expressed in a series of bits, and stores computation results obtained by combining all of the pixel values or all of the indices by associating with the IDs in advance; and
a reading unit that reads the computation result stored in the memory unit using the ID obtained by expressing the plurality of pixel values or the plurality of indices associated with the pixel values in a series of bits.

2. The image processing apparatus according to claim 1, wherein the image processing is to reject noise from an image captured by a camera using a noise rejection filter that performs computation using an input pixel value and a reference pixel value, and
the reading unit reads the computation result stored in the memory unit using the ID obtained by expressing the input pixel value and the reference pixel value input at the time of the noise rejection in a series of bits.

3. The image processing apparatus according to claim 1, wherein an ID obtained by expressing reference information in addition to the input pixel value and the reference pixel value in a series of bits is used.

4. An imaging apparatus comprising:
an excitation light source that irradiates excitation light for exciting a fluorescent dye injected into an examinee onto the examinee;
a camera that obtains a fluorescent image by capturing fluorescence generated from the fluorescent dye by irradiating the excitation light;
an image processing unit that performs image processing to display the fluorescent image obtained by the camera on a display unit, the image processing unit performing noise rejection for an image captured by the camera using a noise rejection filter that performs computation using an input pixel value and a reference pixel value;
a memory unit that sets an ID by expressing the input pixel value and the reference pixel value in a series of bits and stores computation results obtained by combining all of the input pixel values and all of the reference pixel values by associating with the IDs in advance; and
a reading unit that reads the computation result stored in the memory unit using the ID obtained by expressing the input pixel value and the reference pixel value input at the time of the noise rejection in a series of bits.
